SEQUENCE LISTING (6 sequences)

SEQ ID NO: 1 - DNA, 510 bp, Homo sapiens
agagctggcccctgactcacagcccacagagttccacctgctcacaggttggctggctcagccaaggtggtgccctgctctgagcattcaggccaagcccatcctgcaccatggccaggtacagatgctgtcgcagccagagccggagcagatattaccgccagagacaaagaagtcgcagacgaaggaggcggagctgccagacacggaggagagccatgagtaagtgggcccagctgagggtgggctggggctgaggctgggagctctcagggcccagccttcctctcaccacttttcttggtctcaccagggtgctgccgccccaggtacagaccgcgatgtagaagacactaattgcacaaaatagcacatccaccaaactcctgcctgagaatgttaccagacttcaagatcctcttgccacatcttgaaaatgccaccatccaataaaaatcaggagcctgctaaggaacaatgccgcctgtcaataaatgttgaaaagtcatc

SEQ ID NO: 2 - protein, 51 aa, Homo sapiens
MARYRCCRSQSRSRYYRQRQRSRRRRRRSCQTRRRAMRCCRPRYRPRCRRH

SEQ ID NO: 3 - DNA, 20 bp, Artificial
  Primer sequence
agagccgagcagatattacc

SEQ ID NO: 4 - DNA, 20 bp, Artificial
  Primer sequence
tctacatcgcgtctgtacct

SEQ ID NO: 5 - DNA, 20 bp, Artificial
  Primer sequence
ccacccagaagactgtggat

SEQ ID NO: 6 - DNA, 20 bp, Artificial
  Primer sequence
ttctagacggcaggtcaggt

(19) United States
(12) Patent Application Publication
(10) Pub. No.: US 2026/0227405 A1
(43) Pub. Date: Aug. 6, 2026

(54) APPLICATION OF SCTAG IN PREPARATION OF KIT USED TO DIAGNOSE GASTRIC CANCER

(71) Applicant: Jilin University, Changchun City (CN)

(72) Inventor: Fangfang Chen, Changchun (CN)

(73) Assignee: Jilin University, Changchun City (CN)

(21) Appl. No.: 18/037,057

(22) PCT Filed: Oct. 29, 2021
(86) PCT No.: PCT/CN2021/127672

§ 371 Date: Dec. 18, 2023

(30) Foreign Application Priority Data
Nov. 13, 2020 (CN) 202011268995.1

(51) Int. Cl. G01N 33/5753; G01N 33/543; G01N 33/575; G01N 33/68

(52) U.S. Cl. CPC G01N 33/5753; G01N 33/54386; G01N 33/57585; G01N 33/6854; G01N 2333/4703; G01N 2474/20

(57) ABSTRACT

A diagnostic kit for gastric cancer, including antibodies against SCTAG protein, a quality control standard for SCTAG protein, an ELISA kit for diagnosing gastric cancer, and the diagnostic accuracy of gastric adenocarcinoma reached as high as 93.5% by detection of serum SCTAG with the kit. The positive rate of diagnosing early gastric cancer is 96.3%. An immunohistochemical kit for diagnosing gastric cancer with antibody against SCTAG protein as the first antibody, as well as impressive sensitivity and specificity.

### TECHNICAL FIELD

The present invention belongs to the field of biomedical technology and specifically relates to the application of SCTAG in the preparation of a diagnostic kit for gastric cancer.

### SEQUENCE LISTING

This application contains a Sequence Listing that has been submitted electronically as an ASCII text file named “61734-0002001_ST25.txt.” The ASCII text file, created on Feb. 13, 2026, is 2,363 bytes in size. The material in the ASCII text file is hereby incorporated by reference in its entirety.

### BACKGROUND

Gastric cancer is one of the most prevalent malignancies in the world, with over 1000000 new cases and approximately 783000 deaths caused by gastric cancer each year. The early onset symptoms of gastric cancer are extremely insidious, and there is a lack of effective and reliable detection methods for early gastric cancer diagnosis. As a result, most patients are diagnosed at advanced stage, missing the best treatment opportunity and leading to reduced overall survival rate. Detection of serum tumor markers is an optimal way for early detection of asymptomatic tumors. Therefore, searching for tumor biomarkers with high sensitivity and specificity is an important method for improving the diagnosis of early gastric cancer.

Cancer/Testis Antigen (CTA) is considered as tumor associated antigens which only expressed in normal male testicular tissue under physiological conditions, but can be specifically activated in tumor tissue. This unique expression pattern endows CTA with tumor specific expression and high immunogenicity. In recent years, research has shown that CTA also plays an important role in promoting tumor proliferation, invasion, and metastasis. In summary, CTA represents as an unprecedented candidate for tumor diagnosis and immunotherapy. CTA is expressed in various types of tumors, but the expression frequency varies greatly among different tumor types. CT-X antigen is more likely to be expressed in bladder cancer, lung cancer, ovarian cancer and melanoma. However, gastric and colorectal cancer are considered to be CTA poorly-expressed cancer. Gastric cancer specific tumor markers include: serum pepsinogen (PG), Gastrin (G), gastric tumor tissue markers, epidermal growth factor receptor (EGFR), TP53 gene and p53 antibody, mesenchymal epithelial transformation factor (EMT), microsatellite instability (MSI), etc; Novel gastric tumor markers include: microRNA/miR/miRNA, long chain non-coding RNA (lncRNA), matrix metalloproteinase (MMP), etc. In spite of the relatively low frequency of CTA expression in gastric cancer, it is imperative to explore gastric cancer-associated CTAs with specific expression in gastric cancer patients.

SCTAG (super-sensitive Cancer/Testis Antigenfor Gutdiagnosis) is a recently identified CTA, which is expressed in the nucleus of male sperm and involved in sperm chromatin condensation and transcription termination under normal conditions.

### SUMMARY

The purpose of the present invention is to improve the relative low diagnosis efficacy of gastric cancer with tumor markers detection as the main screening method, and to provide an application of SCTAG in the preparation of a diagnostic kit for gastric cancer.

A diagnostic kit for gastric cancer, which includes antibodies against SCTAG;

The described reagent kit for diagnosing gastric cancer has a quality control standard of SCTAG protein (SEQ ID NO: 2);

The described kit for diagnosing gastric cancer is an ELISA kit.

A diagnostic kit for gastric cancer, which includes PCR primers matched with the SCTAG gene (SEQ ID NO: 1);

The PCR described is RT-PCR, qRT-PCR, or real-time PCR;

The primers described are:

(SEQ ID NO: 3)
5′-AGAGCCGAGCAGATATTACC-3′
(SEQ ID NO: 4)
5′-TCTACATCGCGTCTGTACCT-3′;

The described kit for diagnosing gastric cancer also includes reference gene primers:

(SEQ ID NO: 5)
5′-CCACCCAGAAGACTGTGGAT-3′
(SEQ ID NO: 6)
5′-TTCTAGACGGCAGGTCAGGT-3′

An immunohistochemical kit for diagnosing gastric cancer, with its primary antibody being antibody against SCTAG.

The present invention provides a diagnostic kit for gastric cancer, which includes antibodies against SCTAG, SCTAG protein as its quality control standard, and is an ELISA kit. To diagnose gastric adenocarcinoma, the detection rate of SCTAG in patients' serum using this kit can reach as high as 93.5%, and the positive rate of early gastric cancer is 96.3%. The present invention also provides an immunohistochemical reagent kit for diagnosing gastric cancer, with its first antibody being an antibody against SCTAG protein. The reagent kit has been confirmed with high sensitivity and specificity.

### BRIEF DESCRIPTION OF THE DRAWINGS

FIG. 1 shows PCR reaction dissolution curve of SCTAG gene and reference gene GAPDH in gastric cancer, paracancerous, and normal tissues;

FIG. 2 shows DNA agarose gel electrophoresis analysis; g: Internal reference GAPDH, 1: Case 1; 2: Case 2; 3: Case 3; a: Expression of SCTAG-mRNA in gastric cancer tissues; b: Expression of SCTAG-mRNA in gastric cancer adjacent tissues; c: The expression of SCTAG-mRNA in normal gastric tissues;

FIG. 3 shows distribution of SCTAG mRNA expression; (T) Gastric cancer tissues, (P) paracancerous tissues, (N) Nor

FIG. 4 shows analysis of expression frequency of SCTAG mRNA in gastric cancer tissues, paracancerous tissues, and normal tissues.

FIG. 5 shows expression of SCTAG mRNA in human body under physical conditions; M, DNA marker; a, SCTAG mRNA; b, reference gene. 1-14 represent testis, placenta, heart, brain, thyroid, trachea, salivary gland, fetus brain, uterus, colon, stomach, spleen, intestine, bone marrow.

FIG. 6 shows schematic diagram of ratio dilution process for standard.

FIG. 7 shows the correlation between serum SCTAG protein concentrations and clinicopathological features. (A) levels of SCTAG protein in gastric adenocarcinoma were obviously higher than that in healthy control. Correlation between SCTAG protein concentration and clinical variables: (B) age, (C) differentiation level, (D) invasion depth, (E) lymph node metastasis, (F) clinical stage (TNM), (G) tumor size, (H) vascular involvement, (I) nerve involvement.

FIG. 8 shows correlation between SCTAG concentration in gastric adenocarcinoma patients' serum and expression of tumor markers in tumor tissues (A) EGFR, (B) P53, (C) HER-2, (D) Ki67.

FIG. 9 shows evaluation of diagnostic accuracy of serum SCTAG (A) all gastric adenocarcinoma patients, and patients at clinical stage I (B), II (C) and III-IV (D). AUC, area under curve.

FIG. 10 shows comparison of serum SCTAG levels and diagnostic accuracy in male and female patients.

FIG. 11 shows analysis of clinical serological tumor markers in the diagnosis of gastric adenocarcinoma. (A) CEA, (B) CA72-4, (C) CA19-9, (D) CA24-2, (E) CASO, (F) AFP, (G) CEA, CA72-4 and CA19-9, (H) CEA, CA72-4, CA19-9 and CA24-2, (I) CEA, CA72-4, CA19-9, CA24-2, CASO and AFP.

FIG. 12 shows serum SCTAG levels after radical surgery.

FIG. 13 shows expression of SCTAG protein in gastric cancer tissues by IHC. (A) negative control, (B) no expression of SCTAG in normal gastric epithelial tissues, (C) weak positive expression of SCTAG in atypical hyperplasia tissue of stomach, (D-F) strong positive expression of SCTAG in highly, moderately, and poorly differentiated gastric adenocarcinoma.

### DETAILED DESCRIPTION

### Example 1: Total mRNA Extraction and Transcription from Gastric Adenocarcinoma Tissues

### Tissue Sample Collection

Paired gastric adenocarcinoma tissues including cancer tissue and adjacent and normal tissues were selected from the tissue specimen library of the China-Japan Union Hospital of Jilin University (paracancerous tissue is defined as 1.5 cm away from the edge of tumor, and normal tissue is defined as 5 cm away from the edge of tumor). Forty-eight paraffin embedded gastric adenocarcinoma tissues were collected immunohistochemical analysis including cancer tissue, paracancerous tissue, and normal tissue.

Total mRNA Extraction

Use the TRIzol method to extract total mRNA from tissues, as follows:
Take 50 mg of tissue, cut it into small pieces, and place it in 1 mL of TRIzol. Use a dispersion homogenizer to thoroughly crush the tissue and mix well. Allow to stand at room temperature for 5 minutes;
Add 200 μL of chloroform to the above mixture, shake vigorously for 15 seconds to make it fully and evenly mixed, and let it stand for 3 minutes at room temperature;
Centrifuge at 12000 g at 4° C. for 15 minutes, take 450 μL of the upper aqueous phase, and add 450 μL of pre-cooled isopropanol. Mix thoroughly and let stand at room temperature for 10 minutes;
Centrifuge at 12000 g at 4° C. for 10 minutes, and discard the supernatant while retaining the precipitate;
(5) Add 1 mL of 75% ethanol to the precipitate, centrifuge at 7500 g at 4° C. for 5 minutes, and discard the supernatant;
(6) Repeat step 5 once;
(7) Add 30 μL of RNA free (RNase) DEPC water to the precipitate, dissolve it thoroughly at 55° C. for 10 minutes, and place it on ice for subsequent experiments, or store in −80° C. refrigerator;

### 3. Reverse Transcription

Obtain the corresponding total cDNA with mRNA extracted from the above tissue samples by RT-PCR. The specific methods are as follows:
(1) Firstly, concentration detection and quality control were performed on the total mRNA of tissue samples (RNA: 260/280 ratio selection range: 1.9-2.0; 260/230 ratio: 1.8-2.2);
(2) Based on concentration of total mRNA, add lug of mRNA sample to ensure consistency in mRNA loading concentration for each sample, and configure the mixture according to the following system:

| Reagent | Volume(mass) | |
|---|---|---|
| Random Primer | 1 | μL |
| mRNA | 1 | ug |

Add RNase-free DEPC to 15 μL

(3) Incubate at 70° C. for 5 minutes, and then immediately place on ice for 5 minutes;
(4) Prepare the mixture according to the following system and add it to the above reaction system, thoroughly mixing;

| Reagent | Volume | |
|---|---|---|
| 5X M-MLV Buffer | 5 | μL |
| M-MLVreverse transcriptase | 1 | μL |
| dNTP | 1.25 | μL |
| RNase inhibitor | 0.625 | μL |
| RNase-free DEPC | 2.125 | μL |

(5) Place the above mixed reaction system at 42° C. for 60 minutes, then 70° C. for 15 minutes. Remove the cDNA and place on ice for subsequent experiments, or freeze in a −20° C. refrigerator for long storage;

The above steps can be completed using a regular PCR instrument, and obtain 25 μL cDNA.

### 4. Immunohistochemistry Analysis

Reagents: 4% formalin; Paraffin wax; Glass slide; Ethanol aqueous solutions of different concentrations; 3% hydrogen peroxide; TE buffer; 5% goat serum; Anti SCTAG primary antibody; DAB color solution; Hematoxylin dye solution; Sealing agent. The SCTAG protein and monoclonal antibodies Hup1N, Hup1M, and Hup2N were all purchased from Briar patch biosciences.

### Staining Procedures

Slicing: The tissue is embedded in paraffin and a single cancer tissue block is continuously sliced at thickness of 3 μm.

Dewaxing: Bake the paraffin slices on the glass slide at 65° C. for 2 hours. Quickly dewaxing with xylene three times, each for 2 minutes.

Series ethanol rehydration: after dewaxing, the tissue slices are rehydrated with a series of diluted ethanol: anhydrous ethanol for 2 minutes×3 times→95% ethanol for 2 minutes×3 times→80% ethanol for 2 minutesxonce→70% ethanol for 2 minutes×once→PBS flushing for 2 minutes→3 times.

Block endogenous peroxidase: Soak tissue slices with 3% hydrogen peroxide for 10 minutes to quench endogenous peroxides. Rinse with PBS for 2 minutes×3 times.

Antigen repair: Place the slice in a pressure cooker filled with TE buffer (1 mM EDTA and 10 mM Tris, pH 9.2), heat at 98° C. for 30 min, and then rinse it with PBS for 2 min×3 times.

Blocking non-specific proteins: Use an immunohistochemical slicing pen to draw a closed circle 2 mm around the tissue, gently wipe off the liquid around the sample with filter paper, add 5% goat serum (from the same source as the secondary antibody) drop-wise, and place it in a wet box at room temperature for 30 minutes. Do not wash.

Incubation with primary antibody: Shake off goat serum from the section, dry the remaining serum around the tissue with filter paper, and directly add anti SCTAG primary antibody. Add mouse IgG to the control group. Then place it in a wet box on a shaker at 4° C. overnight. Remove from a 4° C. environment and reheat at room temperature for 30 minutes Remove the liquid and rinse with PBS shaker for 5 minutes×3 times.

Secondary antibody incubation: Use filter paper to dry the residual liquid around the tissue, add horseradish peroxidase labeled secondary antibodies, and place them in a wet box. Shake the bed at room temperature for 30 minutes. Remove the secondary antibody liquid and rinse with PBS shaker for 5 minutes×3 times.

DAB color rendering: Use filter paper to dry the residual liquid around the tissue, and add DAB (quickly drop in, observe staining changes, and discard the staining solution). The color reaction time is controlled by observing the color changes under a microscope, and the color reaction time is basically controlled at room temperature for 2-5 minutes. Rinse with tap water and rinse with PBS for 5 minutes×3 times.

Re-staining: Add a large drop of hematoxylin staining solution to the slice, and rinse with tap water after staining until the hematoxylin is completely removed.

Gradual dehydration, transparency, and sealing: Dehydrate in the reverse order of dewaxing and rehydration. After dehydration, immerse the slices in xylene for transparency, and then seal the slices with neutral resin.

Microscopic examination: After the neutral resin has completely dried, place the section under an optical microscope for observation.

### Result Judgment

### Definition of Immunohistochemical Positive Staining

Two pathologists examined the immunohistochemical (IHC) staining results without understanding clinical data, and each independently read the results of tissue slices at least twice. After comparing the results, they reread the slices whenever there were differences in the results until a consensus was reached. In short, IHC staining is semi quantitatively labeled as “−” (negative: no or less than 5% positive cells), “+” (6-25% positive cells), “++” (26-50% positive cells), and “+++” (more than 50% positive cells). Finally, the three groups of “+”, “++”, and “+++” were classified as IHC staining positive groups;—“is classified as the IHC staining negative group.

### Definition of Immunohistochemical Intensity

Analyze the intensity of positive staining in tissues with Integrated Optical Density (IOD) using Image-Pro Plus software (version 6.0). Select 10 digital images for each sample under the same optical microscope and exposure intensity. After the Image Pro Plus color collection, calculate the total IOD value of positive staining and the total area of positive staining Area (μm²). Take the IOD/Area ratio of each image to represent the staining intensity of that image. Finally, take the average IOD/Area ratio of 10 digital images as the numerical value of the staining intensity for this slice.

### Sample 2 Analysis of SCTAG Expression in Gastric Adenocarcinoma Tissues

### Design and Synthesis of Primers

Design forward and reverse oligonucleotide primers for SCTAG and GAPDH sequences using Primer Express version 3.0 software, and synthesize by Shanghai Sangon Company; according to the content of different primers, the primer concentration is ultimately diluted to the working concentration (10 μM). The primer sequence information is as follows (Table 1):

TABLE 1
Primers of GAPDH and SCTAG

| Gene | Primers | Sequences | Length |
|---|---|---|---|
| GAPDH | Forward | CCACCCAGAAGACTGTGGAT | 203 bp |
| | | (SEQ ID NO: 5) | |
| | Reverse | TTCTAGACGGCAGGTCAGGT | |
| | | (SEQ ID NO: 6) | |
| SCTAG | Forward | AGAGCCGAGCAGATATTACC | 119 bp |
| | | (SEQ ID NO: 3) | |
| | Reverse | TCTACATCGCGTCTGTACCT | |
| | | (SEQ ID NO: 4) | |

Preparation of qRT-PCR reaction system

### Add the Following Reaction Systems to the POR Octuple

| Reagent | Volume | |
|---|---|---|
| 2X GoTaq ®qPCR Master Mix | 10 | μL |
| F-primer (10 μM) | 0.4 | μL |
| R-primer (10 μM) | 0.4 | μL |
| cDNA | 1 | μL |
| DEPC | 8.2 | μL |

React and amplify the reaction system after mixing according to the following qPCR amplification procedure. Complete a total of 40 cycles:

| | | |
|---|---|---|
| Temperature | Time | Cycle |

| | | | |
|---|---|---|---|
| 95° C. | 10 | min | 1 |
| 95° C. | 15 | s | |
| 60° C. | 1 | min | 40 |

Melting curve analysis

Make three repeats for each sample, and take the average Ct (Cycle threshold) value as the Ct value of the sample.

### Calculation of Ct Value

The expression level of a target gene is determined by ΔCt value, ΔCt=Ct (target gene: SCTAG)-Ct (internal reference gene: GAPDH). Relative expression in cancer or paracancerous tissue is represented by −ΔΔCt (ΔCt in normal tissue minus ΔCt in paired paracancerous or cancer tissue). If −ΔΔCt>O, it indicates that the expression level of SCTAG mRNA in paracancerous/cancer tissues is higher than that in normal tissues.

### Expression of SCTAG in Gastric Adenocarcinoma Tissues

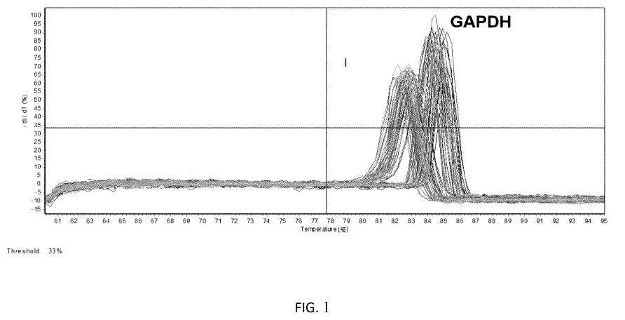

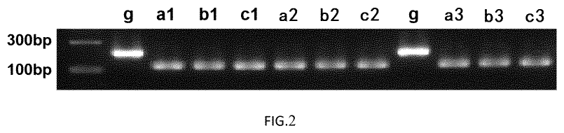

Using Primer Express version 3.0 software, primers were designed for SCTAG and GAPDH, and the melting curve showed that their dissolution peak was in a single peak state (FIG. 1); This indicates that these primer designs are reasonable and have good singularity; The electrophoresis results also showed that the expression band length of SCTAG met the expected fragment length of the primer design (FIG. 2); And the qPCR product was sent to Shanghai Sangon Company for sequencing, and the sequence obtained was consistent with the sequence of the target gene.

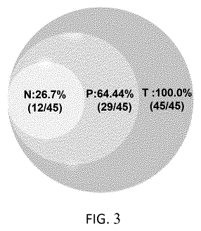

SCTAG mRNA was detected in a total of 45 gastric cancer tissues (93.75%, 45/48); SCTAG mRNA expression was also detected in paracancerous tissues (60.42%, 29/48) and normal tissues (25.00%, 12/48); The chi square test results showed that the closer to the center of the tumor, the higher the positive frequency of SCTAG mRNA expression (p<0.001) (Table 2). At the same time, the experiment also found that whenever SCTAG mRNA was expressed in normal tissues, it was also detected in the paired cancer tissues and paracancerous tissues; whenever SCTAG mRNA was expressed in paracancerous tissues, it was also detected in paired cancer tissues but not always in normal tissues. In one word, SCTAG mRNA expression in cancer tissues did not predict the expression in paired paracancerous and normal tissues (FIG. 3).

TABLE 2
Expression of SCTAG mRNA in paired
gastric adenocarcinoma tissues

| Tissues | NO | Negative | Positive | χ² | P-value |
|---|---|---|---|---|---|
| Normal tissue | 96 | 100% | 0% | 148.8 | <0.001 ※ |
| | | (96/96) | (0/96) | | |
| Atypical | 86 | 38.4% | 61.6% | | |
| hyperplasia | | (33/86) | (53/86) | | |
| tissue | | | | | |
| Gastric | 120 | 18.3% | 81.7% | | |
| adenocarcinoma | | (22/120) | (98/120) | | |
| tissue | | | | | |

※ □P < 0.001, difference is significant.

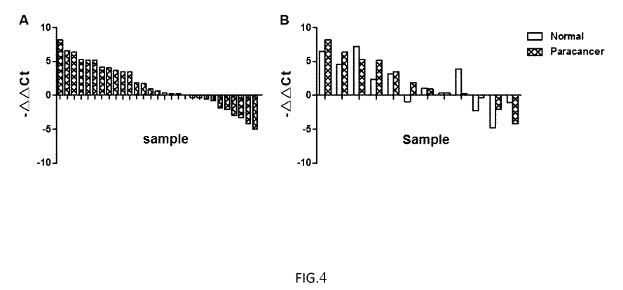

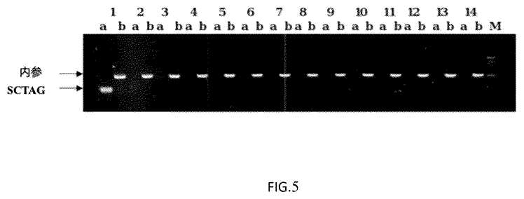

The expression level of SCTAG mRNA also varies among different tissues. Among 29 gastric cancer patients who expressed SCTAG mRNA in both cancer and adjacent tissues, the expression level of SCTAG mRNA in adjacent tissues was generally higher than that in cancer tissues (FIG. 4A); In 12 patients who expressed SCTAG mRNA in cancer, paracancerous, and normal tissues, the expression level of SCTAG mRNA was in order of paracancerous tissue>normal>tumor tissue (FIG. 4B). In summary, the expression level of SCTAG mRNA in paracancerous tissues is higher than that in normal and tumor tissues, indicating that SCTAG plays a role in cell differentiation and tumorigenesis. FIG. 5 shows the expression results of SCTAG mRNA in healthy human testicular tissue. The results showed that SCTAG mRNA was only expressed in testicular tissue and not in other tissues and organs in healthy individuals.

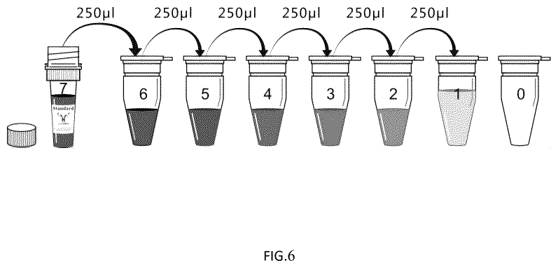

Sample 3 Detection of Serum SCTAG Procedures are as follows:
1. Move the ELISA kit, sample diluent, and other reagents to room temperature (18-25° C.) and equilibrate for at least 30 minutes; Centrifuge the standard for 30-60 seconds, add 1 mL of sample diluent and fully dilute to 2000 μg/mL with the standard stock solution. After dilution, balance for at least 10 minutes;
2. Standard dilution ratio: as shown in FIG. 6, transfer 250 μL dilute sample into each test tube (numbered 0-6); Suck 250 μL standard stock solution to tube No. 6, thoroughly blow and mix. After thorough mixing, draw 250 μL of mixed solution from tube No. 6 and add it to tube No. 5. Transfer it to tube No. 1 in sequence. The undiluted standard solution is used as the standard with the highest concentration (2000 pg/mL). The sample dilution is used as a zero concentration standard (Opg/ml);
3. Sample dilution: The collected serum samples were diluted with sample diluent in a ratio of 1:5;
4. Sampling: Add 100 μL standard solution or sample to be tested to each hole with a platoon gun, with double secondary holes for the standard hole and three secondary holes for the sample hole to be tested; Gently shake and mix well, cover the upper plate and incubate at 37° C. for 2 hours;
5. Discard the liquid, invert the plate and shake it dry, and use a clean tissue to absorb it without washing;
6. Add 100 μL biotin labeled antibody to each hole. Cover with new board stickers. Incubate at 37° C. for 1 hour;
7. Discard the liquid in the hole, shake it dry, and add cleaning buffer 200 μL to each hole and soak for 2 minutes each time. Wash the board 3 times in total;
8. Add 100 μL horseradish peroxidase labeled avidin (HRP avidin protein) to each hole. Cover with a new board sticker. Incubate at 37° C. for 1 hour;
9. Repeat steps 7-8, washing the board 5 times in total;
10. Add TMB substrate solution 90 μL to each hole. Incubate at 37° C. for 15-30 minutes, avoiding light for color rendering. (Reaction time control: When there is a blue change in hole number 1 (31.25 pg/mL), but no blue change in hole number 0 (0 pg/mL), the reaction is terminated);
11. Add termination liquid 50 μL to each hole, gently tap the drip plate to ensure full mixing;
12. Measure the absorbance values (OD values) of each well at a wavelength sequence of 450 nm within 5 minutes after the termination of the reaction;
13. Calculation and analysis of ELISA data results: Calculate the average absorbance OD values of the standard and sample wells separately, and the detection value of a single well should not exceed 20% of the average value; Subtract the OD value of well number 0 (concentration Opg/mL) from the average value of each standard and sample; Establish a standard curve using the absorbance OD value of the standard as the x-axis (X) and the corresponding SCTAG protein concentration of the standard as the y-axis (Y); The SCTAG protein content of the sample to be tested can be calculated by the standard curve based on its OD value; The final actual concentration of the sample should be the concentration obtained on the standard curve multiplied by the dilution factor.
14. Correlation between serum SCTAG and clinicopathological features of gastric cancer patients

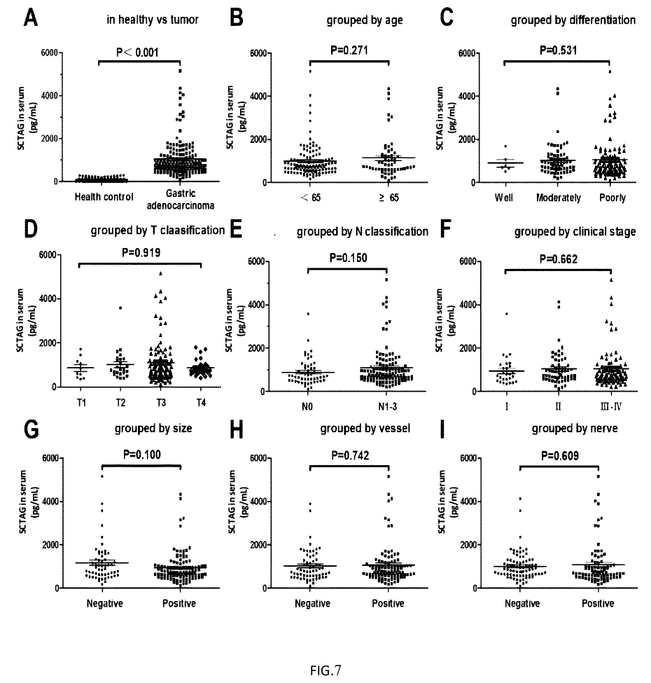

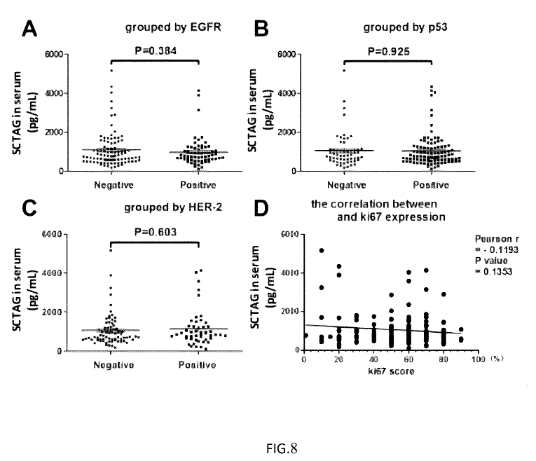

Compared to healthy controls, serum SCTAG in patients were significantly higher (94.30±88.70 pg/mLvs 1040.31±826.52 pg/mL) (FIG. 7A). Besides, serum SCTAG levels in patients were not associated with clinicopathological features, including age (P=0.271), differentiation level (P=0.531), infiltration dept (P=0.919), lymph node metastasis (P=0.150), clinical stage (P=0.662), tumor size (P=0.100), vascular involvement (P-0.742), and nerve involvement (P=0.609). Moreover, serum SCTAG was not associated with expression level of EGFR (P=0.384), P53 (P=0.925), HER-2 (P=0.603), and Ki-67 (P=0.1353) (FIG. 8).

### Sample 4 Evaluation of Serum SCTAG in the Diagnosis of Gastric Cancer.

### Diagnosis of Early Gastric Cancer

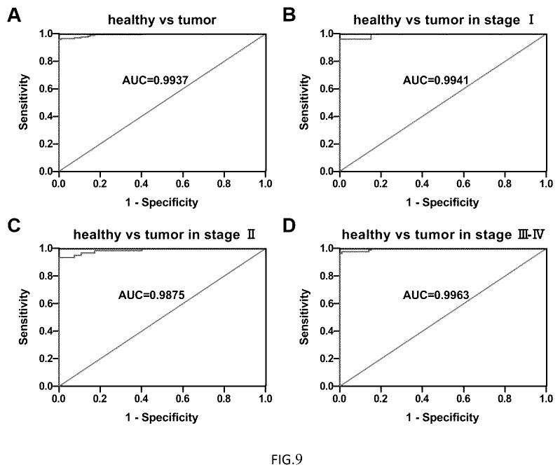

By establishment of ROC curve, the area under the ROC curve (AUC) was 0.9937 (FIG. 9A), which showed that serum SCTAG protein had high sensitivity and specificity for the diagnosis of gastric adenocarcinoma patients. Meanwhile, in order to evaluate the diagnostic accuracy of serum SCTAG protein for early gastric cancer, we conducted further analysis on three clinical variables: clinical stage I, clinical stage II, and clinical stage III-IV. We found that among gastric adenocarcinoma patients at clinical stage III-IV, the diagnostic accuracy of serum SCTAG protein was the highest (AUC=0.9963, FIG. 9D); Moreover, serum SCTAG protein also has high diagnostic accuracy in early gastric cancer (clinical stage I) (AUC=0.9941, FIG. 9B). Taken mean plus 3-fold standard deviation (Mean+3SD) of serum SCTAG in the healthy control group as the diagnostic criteria, the positive rate of serum SCTAG in the diagnosis of gastric adenocarcinoma patients was as high as 93.5% (159/170), and the positive rate of early gastric cancer patients (clinical stage I) was 96.3% (26/27).

### Diagnostic Accuracy Between Male and Female

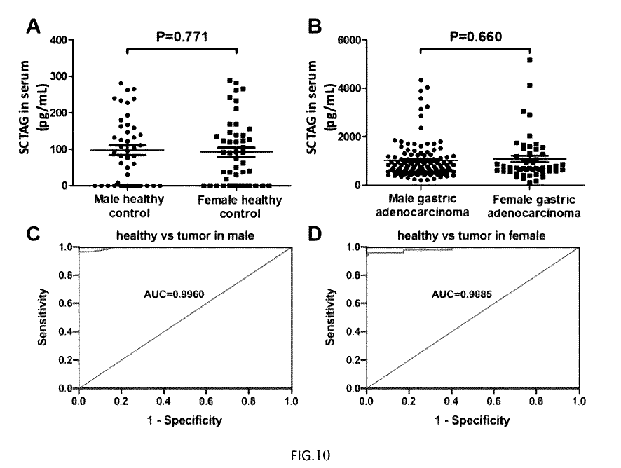

Under normal conditions, SCTAG is only present in males (testicular region). We compared the differences in serum SCTAG concentrations between male and female in healthy control group and gastric cancer group to confirm whether SCTAG can be used as a serum tumor biomarker for both male and female populations. The results showed that there was no difference in serum SCTAG concentration between the male (97.08+89.60 pg/mL) and female (91.64+88.7 lpg/mL) healthy control groups (P=0.771) (FIG. 10A). At the same time, there was no difference in serum SCTAG concentration between male gastric adenocarcinoma patients (1021.99+771.74 pg/mL) and female gastric adenocarcinoma patients (1083.05=949.09 pg/mL) (P=0.660) (FIG. 10B). Furthermore, we established ROC curves and analyzed AUC. The results showed that the AUC value for males was 0.9960 (FIG. 10C), while the AUC value for females was 0.9885 (FIG. 10D), indicating a similar diagnostic accuracy for serum SCTAG. These data all indicate that the diagnostic accuracy of serum SCTAG for gastric adenocarcinoma is not affected by gender, and can be applied as a potential serum marker for diagnosing gastric adenocarcinoma suitable for a wide range of population screening.

Comparison of Serum SCTAG with Other Serological Markers

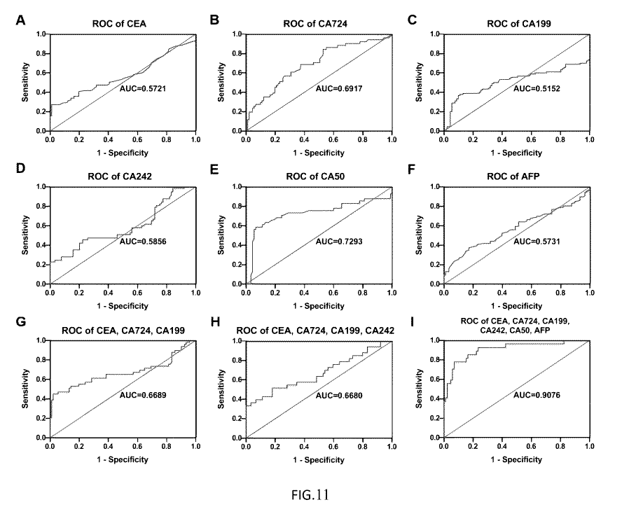

We further compared the diagnostic accuracy of other clinical tumor markers in gastric cancer patients, and found that both CA724 (AUC=0.6917) and CA50 (AUC=0.7293) had moderate diagnostic accuracy (FIG. 11B, E). However, the diagnostic accuracy of other tumor markers is relatively poor, such as CEA (AUC-0.5721), CA199 (AUC=0.5152), CA242 (AUC=0.5856), AFP (AUC=0.5731) (FIG. 11A, C, D, F). For gastric cancer, which is difficult to screen, joint detection of multiple indicators can effectively improve the diagnosis of gastric cancer. The diagnostic accuracy of three markers panel (CEA, CA199, and CA724) and four markers panel (CEA, CA724, CA199, and CA242) improved to AUC=0.6689 and AUC=0.6680, respectively (FIG. 11G, H). The diagnostic accuracy of six markers panel for gastrointestinal tumors can reach as high as 0.9076 (FIG. 11I). However, the diagnostic accuracy of these panels did not exceed that of serum SCTAG, indicating that SCTAG protein has stronger diagnostic ability for gastric adenocarcinoma with impressive sensitivity and specificity.

Tendency of Serum SCTAG after Radical Surgery

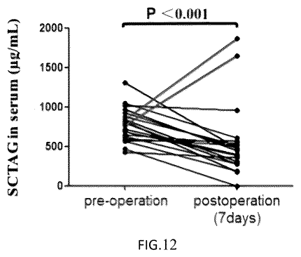

In gastric adenocarcinoma patients, 18 of them received radical gastroectomy, and 2 underwent switch surgery due to extensive metastasis in the abdominal cavity. The serum SCTAG in patients undergoing radical gastroectomy significantly reduced from 788.11±214.34 pg/mL before surgery to 415.05±207.33 pg/mL after surgery (P<0.001) within 7 days. However, serum SCTAG in gastric cancer patients who underwent switch surgery increased from 801.78±47.39 pg/mL before surgery to 1757.47±160.29 pg/mL after surgery (FIG. 12). This further implied that serum SCTAG level was associated with tumor burden in gastric cancer patients which can be used for postoperative monitoring of the presence of gastric tumor cells and real-time detection of the presence of gastric adenocarcinoma.

### Expression of SCTAG in Gastric Adenocarcinoma Tissues

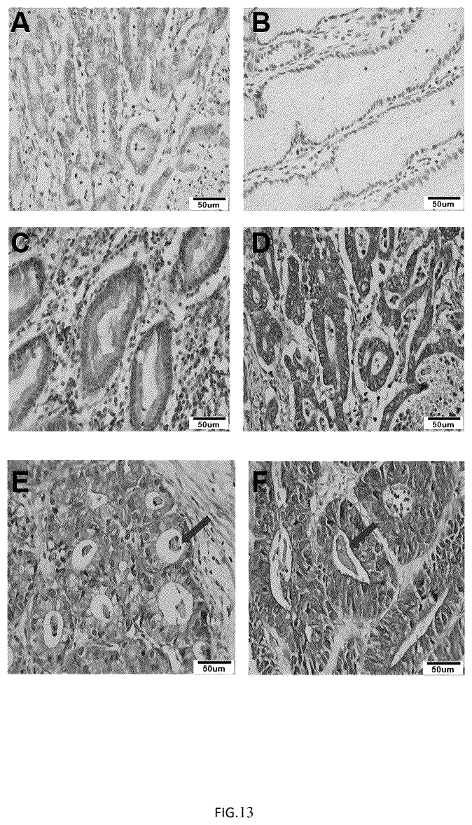

We noticed that SCTAG protein is widely expressed in gastric adenocarcinoma tissues (98/120, 81.7%) (Table 2). Immunohistochemical staining showed that SCTAG protein was distributed in the cytoplasm of gastric adenocarcinoma tissues (FIG. 12). However, no expression of SCTAG protein was found in normal tissues (0/96, 0%) (Table 2; FIG. 13B). Interestingly, we also found that SCTAG protein was expressed in a small amount of atypical hyperplasia tissues of stomach with a positive rate at 61.6% (Table 2; FIG. 13C). The expression frequency of SCTAG protein showed an increasing tendency in gastric adenocarcinoma, atypical hyperplasia, and normal tissues (P<0.001).

SCTAG protein expression in gastric adenocarcinoma tissues is highly correlated with the degree of cellular differentiation (Table 3, P=0.004). Specifically, the positive rate of SCTAG protein expression was 100% in high differentiated tissues, 93.2% in medium differentiated tissues, and 71.2% in poor differentiated tissues (Table 3). Meanwhile, the positive rate of SCTAG expression was significantly correlated with clinical staging of gastric cancer (Table 3, p=0.023). The higher the clinical stage was, the higher frequency of SCTAG expression was observed in gastric adenocarcinoma patients. SCTAG protein is also more likely to be expressed in tumors with vascular involvement (P=0.044) and nerve involvement (P-0.033). The expression frequency of SCTAG in gastric adenocarcinoma tissue was not associated with patients' age (P=0.94), gender (P=0.119), invasion depth (T stage) (P=0.064), lymph node metastasis (P=0.073), gross classification (P=0.744), and tumor size (P=0.082) (Table 3).

Meanwhile, SCTAG expression in gastric cancer tissue was not significantly correlated with the expression levels of tumor markers EGFR (P=0.329), p53 (P=0.544), and HER-2 (P=0.474) in gastric tissue (Table 3). Additionally, we also noticed the positive staining of SCTAG protein in gland lumen of gastric adenocarcinoma, which implied the secretion of SCTAG from gastric cancer cells (pointed by black arrow in FIG. 13).

TABLE 3
Correlation between SCTAG protein in gastric adenocarcinoma
tissues and clinicopathological features

| | | SCTAG | | | |
|---|---|---|---|---|---|
| Variables | NO | Negative(%) | Positive (%) | χ² | P-value |
| Age | | | | 0.006 | 0.94 |
| <65 | 81 | 15 (18.5%) | 66 (81.5%) | | |
| ≥65 | 39 | 7 (17.9%) | 32 (82.1%) | | |
| Gender | | | | 2.429 | 0.119 |
| Male | 87 | 13 (14.9%) | 74 (85.1%) | | |
| Female | 33 | 9 (27.3%) | 24 (72.7%) | | |
| Differentiation | | | | 10.96 | 0.004^{※} |
| level | | | | | |
| Well | 10 | 0 (0.0%) | 10 (100.0%) | | |
| Moderate | 44 | 3 (6.8%) | 41 (93.2%) | | |
| Poor | 66 | 19 (28.8%) | 47 (71.2%) | | |
| T stage | | | | 3.428 | 0.064 |
| 1-2 | 26 | 8 (30.8%) | 18 (69.2%) | 3.210 | 0.073 |
| 3-4 | 94 | 14 (14.9%) | 80 (85.1%) | | |
| Lymph node | | | | | |
| metastasis | | | | | |
| Negative | 39 | 10 (25.6%) | 29 (74.4%) | | |
| Positive | 81 | 12 (14.8%) | 69 (85.2%) | | |
| Clinical stage | | | | 7.530 | 0.023※ |
| I | 20 | 8 (40.0%) | 12 (60.0%) | | |
| II | 49 | 7 (14.3%) | 42 (85.7%) | | |
| III-IV | 51 | 7 (13.7%) | 44 (86.3%) | | |
| Gross | | | | 0.106 | 0.744 |
| classification | | | | | |
| Protruded | 18 | 3 (16.7%) | 15 (83.3%) | | |
| Ulcer | 90 | 18 (20.0%) | 72 (80.0%) | | |
| Tumor size | | | | 3.028 | 0.082 |
| (cm) | | | | | |
| ≤4 | 37 | 10 (27.0%) | 27 (73.0%) | | |
| >4 | 80 | 11 (13.8%) | 69 (86.2%) | | |
| Vascular | | | | 4.042 | 0.044^{※} |
| involvement | | | | | |
| Negative | 52 | 14 (26.9%) | 38 (73.1%) | | |
| Positive | 65 | 8 (12.3%) | 57 (87.7%) | | |
| Nerve | | | | 4.530 | 0.033^{※} |
| involvement | | | | | |
| Negative | 58 | 15 (25.9%) | 43 (74.1%) | | |
| Positive | 57 | 6 (10.5%) | 51 (89.5%) | | |
| EGFR | | | | 0.954 | 0.329 |
| Negative | 72 | 15 (20.8%) | 57 (79.2%) | | |
| Positive | 44 | 6 (13.6%) | 38 (86.4%) | | |
| p53 | | | | 0.368 | 0.544 |
| Negative | 43 | 9 (20.9%) | 34 (79.1%) | | |
| Positive | 73 | 12 (16.4%) | 61 (83.6%) | | |
| HER-2 | | | | 0.513 | 0.474 |
| Negative | 56 | 9 (16.1%) | 47 (83.9%) | | |
| Positive | 37 | 4 (10.8%) | 33 (89.2%) | | |

What is claimed is:

1. A diagnostic kit for gastric cancer, including antibody against SCTAG.

2: A diagnostic kit of claim 1, wherein its quality control standard is SCTAG protein.

3: A diagnostic kit of claim 1, wherein the diagnostic kit for gastric cancer is an ELISA kit.

4: An immunohistochemical kit for diagnosing gastric cancer, wherein its first antibody is antibody against SCTAG.

5. A diagnostic kit of claim 2, wherein the diagnostic kit for gastric cancer is an ELISA kit.
